(19) United States
(12) Patent Application Publication
(10) Pub. No.: US 2025/0082385 A1
(43) Pub. Date: Mar. 13, 2025

(54) MULTIPLE GAS CIRCUIT CONNECTOR AND METHOD FOR CRYOABLATION SYSTEM

(71) Applicant: Boston Scientific Scimed, Inc., Maple Grove, MN (US)

(72) Inventors: Kyle True, Minneapolis, MN (US); Eric T. Gagner, Minnetonka, MN (US); Timothy A. Ostroot, Cokato, MN (US); Cory Ross Stenberg, St. Michael, MN (US); Benjamin Wai-Man Chan, Edina, MN (US); Zachary Nickle, Minneapolis, MN (US)

(21) Appl. No.: 18/671,677

(22) Filed: May 22, 2024

Related U.S. Application Data
(60) Provisional application No. 63/537,324, filed on Sep. 8, 2023.

(51) Int. Cl. A61B 18/02; A61B 18/00

(52) U.S. Cl. CPC A61B 18/02; A61B 2018/00101; A61B 2018/00172; A61B 2018/00577; A61B 2018/0091; A61B 2018/0212; A61B 2018/0262

(57) ABSTRACT

In an embodiment, a cryoablation system includes a pre-cooler gas circuit, a working gas circuit isolated from the pre-cooler gas circuit, and a vacuum chamber isolated from the pre-cooler gas circuit and the working gas circuit. The cryoablation system can include a shaft having an insulated zone and a working gas expansion chamber distal to the insulated zone. The cryoablation system can further include a handle and a shaft-handle connector, wherein a proximal end of the shaft connects to the shaft-handle connector, wherein the shaft-handle connector is configured to removably attach the proximal end of the shaft to a distal end of the handle.

### RELATED APPLICATIONS

This application claims the benefit of U.S. Provisional Application No. 63/537,324, filed Sep. 8, 2023, the content of which is herein incorporated by reference in its entirety.

### TECHNICAL FIELD

Embodiments herein relate to cryoablation systems and more particularly to cryoablation systems with detachable shafts.

### BACKGROUND

During cryosurgery, a surgeon may deploy one or more cryoprobes to ablate a target area of a patient anatomy by freezing and thawing the tissue. In one example, a cryoprobe uses the Joule-Thomson effect to produce cooling or heating of the probe tip. In such cases, the expansion of a cryofluid in the cryoablation probe from a higher pressure to a lower pressure leads to cooling of the device tip to temperatures at or below those corresponding to cryoablation in tissue in the vicinity of the tip. Heat transfer between the expanded cryofluid and the outer walls of the cryoprobe leads to formation of an ice ball in the tissue around the tip and consequent cryoablation of the tissue.

### SUMMARY

In a first aspect, a cryoablation system can include a pre-cooler gas circuit, a working gas circuit, and a vacuum chamber the working gas circuit. The cryoablation system can include a shaft having an insulated zone along a proximal length of the shaft. The insulated zone can include a vacuum chamber shaft portion and an insulated portion of the working gas circuit, wherein the vacuum chamber shaft portion surrounds and can be isolated from the insulated portion of the working gas circuit. The shaft can include a working gas expansion chamber distal to the insulated zone, wherein the working gas expansion chamber includes an expansion portion of the working gas circuit. The cryoablation system can include a handle having a handle portion of the vacuum chamber, and a handle portion of the working gas circuit. The cryoablation system can further include a shaft-handle connector. A proximal end of the shaft can connect to the shaft-handle connector and the shaft-handle connector can be configured to removably attach the proximal end of the shaft to a distal end of the handle.

In a second aspect, in addition to one or more of the preceding or following aspects, or in the alternative to some aspects, the shaft includes a supply tube extending along a portion of a length of the shaft, wherein the supply tube can be surrounded by a return tube along a portion of a length of the supply tube, wherein the return tube can be surrounded by an insulating shaft along the insulated zone of the shaft, wherein the shaft-handle connector can be configured to form a seal around an outer surface the insulating shaft.

In a third aspect, in addition to one or more of the preceding or following aspects, or in the alternative to some aspects, the shaft-handle connector includes a first connector piece and a second connector piece, wherein a protrusion of the second connector piece can be configured to extend within a cavity defined within the first connector piece.

In a fourth aspect, in addition to one or more of the preceding or following aspects, or in the alternative to some aspects, an inner surface of the protrusion of the second connector piece of the shaft-handle connector can be configured to form a seal around an outer surface of the return tube.

In a fifth aspect, in addition to one or more of the preceding or following aspects, or in the alternative to some aspects, the second connector piece of the shaft-handle connector includes an interior space, and an inner surface of the interior space can be configured to form a seal around an outer surface of the supply tube.

In a sixth aspect, in addition to one or more of the preceding or following aspects, or in the alternative to some aspects, an inner surface of the handle can be configured to form a seal around an outer surface of the shaft-handle connector.

In a seventh aspect, in addition to one or more of the preceding or following aspects, or in the alternative to some aspects, the shaft can be removed from the handle without causing any damage to an ability of the handle to isolate the handle portion of the working gas circuit and isolate the handle portion of the vacuum chamber.

In an eighth aspect, in addition to one or more of the preceding or following aspects, or in the alternative to some aspects, the shaft-handle connector includes a connector portion of the vacuum chamber, wherein the shaft-handle connector defines one or more openings in fluid communication with the connector portion of the vacuum chamber and configured to connect to a vacuum chamber portion of the handle.

In a ninth aspect, in addition to one or more of the preceding or following aspects, or in the alternative to some aspects, the shaft-handle connector defines one or more openings through which a return portion of the working gas circuit runs between the handle and the shaft-handle connector.

In a tenth aspect, in addition to one or more of the preceding or following aspects, or in the alternative to some aspects, the cryoablation system further comprises a pre-cooler gas circuit isolated from the working gas circuit and the vacuum circuit, wherein the handle comprises a handle portion of the pre-cooler gas circuit. The pre-cooler gas circuit can be configured to supply a pre-cooler gas from a high-pressure cryogenic gas source to the handle and the pre-cooler gas circuit can include a pre-cooler Joule-Thomson orifice where the pre-cooler gas enters a pre-cooler expansion chamber.

In an eleventh aspect, in addition to one or more of the preceding or following aspects, or in the alternative to some aspects, the working gas circuit can be configured to supply a working gas from a high-pressure cryogenic gas source to the working gas expansion chamber, the working gas circuit can include a working gas Joule-Thomson orifice where the working gas enters the working gas expansion chamber.

In a twelfth aspect, a cryoablation system includes a working gas circuit and a vacuum chamber isolated from the working gas circuit. The cryoablation system can include a shaft having an insulated zone along a proximal length of the shaft, the insulated zone having a vacuum chamber shaft portion and an insulated portion of the working gas circuit, wherein the vacuum chamber shaft portion surrounds and can be isolated from the insulated portion of the working gas circuit. The shaft can include a working gas expansion chamber distal to the insulated zone including an expansion portion of the working gas circuit. The cryoablation system can include a shaft-handle connector. A proximal end of the shaft can connect to the shaft-handle connector and the shaft-handle connector can be configured to removably attach the proximal end of the shaft to a distal end of a handle. The shaft-handle connector further includes a working gas connector structure configured to make a sealed connection to a working gas supply passage in the handle and a working gas exhaust passage in the handle, and vacuum connector structure configured to make a sealed connection to a vacuum chamber portion of the handle, and a connector portion of the vacuum chamber isolated from a connector portion of the working gas circuit.

In a thirteenth aspect, in addition to one or more of the preceding or following aspects, or in the alternative to some aspects, the shaft includes a supply tube extending along a portion of a length of the shaft, wherein the supply tube can be surrounded by a return tube along a portion of a length of the supply tube, wherein the return tube can be surrounded by an insulating shaft along the insulated zone of the shaft, wherein the shaft-handle connector can be configured to form a seal around an outer surface the insulating shaft.

In a fourteenth aspect, in addition to one or more of the preceding or following aspects, or in the alternative to some aspects, the shaft-handle connector includes a first piece and a second piece, wherein a protrusion of the second piece can be configured to extend within a cavity defined within the first piece.

In a fifteenth aspect, in addition to one or more of the preceding or following aspects, or in the alternative to some aspects, an inner surface of the protrusion of the second piece of the shaft-handle connector can be configured to form a seal around an outer surface of the return tube.

In a sixteenth aspect, in addition to one or more of the preceding or following aspects, or in the alternative to some aspects, the second piece of the shaft-handle connector includes an interior space, and an inner surface of the interior space can be configured to form a seal around an outer surface of the supply tube.

In a seventeenth aspect, in addition to one or more of the preceding or following aspects, or in the alternative to some aspects, the shaft-handle connector includes a connector portion of the vacuum chamber, wherein the shaft-handle connector defines one or more openings in fluid communication with the connector portion of the vacuum chamber and configured to connect to a vacuum chamber portion of the handle.

In an eighteenth aspect, in addition to one or more of the preceding or following aspects, or in the alternative to some aspects, the shaft-handle connector defines one or more openings through which a return portion of the working gas circuit runs between the handle and the shaft-handle connector.

In a nineteenth aspect, in addition to one or more of the preceding or following aspects, or in the alternative to some aspects, the working gas circuit can be configured to supply a working gas from a high-pressure cryogenic gas source to the working gas expansion chamber, the working gas circuit can include a working gas Joule-Thomson orifice where the working gas enters the working gas expansion chamber.

In a twentieth aspect, a method of operating a cryoablation system can include providing a cryoablation system. The cryoablation system can include a working gas circuit. The cryoablation system can include a first catheter having a first shaft and a first shaft-handle connector. The first shaft can include a first working gas expansion chamber. The cryoablation system can include a handle having a handle portion of the working gas circuit. A proximal end of the first shaft can connect to the first shaft-handle connector and the first shaft-handle connector removably attaches the proximal end of the first shaft to a distal end of the handle. The method can include detaching the first catheter assembly from the handle. The method can include attaching a second catheter assembly to the handle. The second catheter assembly includes a second shaft and a second shaft-handle connector, the second shaft can include a second working gas expansion chamber, wherein a proximal end of the second shaft connects to the second shaft-handle connector. The second shaft-handle connector can be configured to removably attach the proximal end of the second shaft to a distal end of the handle.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cryoablation system in accordance with various embodiments herein.

FIG. 2 is a schematic view of portions of a cryoablation system in accordance with various embodiments herein.

FIG. 3 is a schematic view of a portion of a cryoablation shaft shown in accordance with various embodiments herein in accordance with various embodiments herein.

FIG. 4 is a cross sectional view of the shaft of FIG. 3 taken along section 4-4 in FIG. 3 in accordance with various embodiments herein.

FIG. 5 is a cross-sectional view of the shaft of FIG. 3 taken along section 5-5 in FIG. 3 in accordance with various embodiments herein.

FIG. 6 is a schematic view of a cryoablation system in accordance with various embodiments herein.

FIG. 7 is a cross-sectional view of the cryoablation system of FIG. 6 along line 7-7 in FIG. 6 in accordance with various embodiments herein.

FIG. 8 is a closeup view of the cryoablation system of FIG. 7 about detail 8 in FIG. 7 in accordance with various embodiments herein.

FIG. 9 is a side view of a shaft-handle connector in accordance with various embodiments herein.

FIG. 10 is a cross sectional view of the shaft-handle connector in accordance with various embodiments herein.

FIG. 11 is an exploded side view of the shaft-handle connector in accordance with various embodiments herein.

FIG. 12 is a side view of the catheter assembly in accordance with various embodiments herein.

FIG. 13 is a cross sectional view of the catheter assembly in accordance with various embodiments herein.

FIG. 14 is a flowchart describing a method for using a cryoablation system in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Cryoablation, also known as cryotherapy or cryosurgery, is a medical procedure that involves using extreme cold temperatures to destroy or remove abnormal or diseased tissue. Cryoablation is used in various medical fields, including oncology (cancer treatment), cardiology (heart treatment), dermatology (skin treatment), and more. In cryoablation, a shaft is inserted into or near the targeted tissue. This shaft contains a cryogenic substance, such as liquid nitrogen or argon gas, which is used to rapidly cool the tissue to very low temperatures. The extreme cold causes ice crystals to form within the cells, leading to cellular damage and eventual cell death.

In some applications, cryoablation systems use a rigid shaft to deliver the cryogenic substance to the target anatomy. Rigid shafts are generally more robust but offer limited access to a patient's anatomy. Some cryoablation systems may be useful for ablating lesions in the biliary system or other difficult to access portions of the human anatomy. To access such anatomical features, a flexible cryoablation shaft can be implemented. However, it is more challenging for flexible shafts to contain high-pressure gas.

In most cases, after a cryoablation procedure is performed, the catheter of a cryoablation system is considered a one-use item and is designed to be removed and replaced. It is desirable to reuse other portions of the cryoablation system where possible, such as the handle and control console, over multiple cryoablation procedures.

The present disclosure is directed towards a cryoablation system having a detachable catheter assembly. The catheter assembly can include a shaft and a shaft-handle connector. The shaft may be removably attached to the handle using a shaft-handle connector. The shaft-handle connector allows for the shaft of a cryoablation system to be replaced while keeping the multiple fluid circuits (e.g., pre-cooler gas, working gas, and vacuum) isolated from one another within the handle.

The concepts described herein can be applied in the context of the cryoablation systems described in US Published Patent Application US2021/00045793, titled “Dual Stage Cryocooler,” and US Published Patent Application US2021/00045794, titled “Flexible Cryoprobe,” both filed Aug. 14, 2020, and both incorporated herein by reference in their entireties.

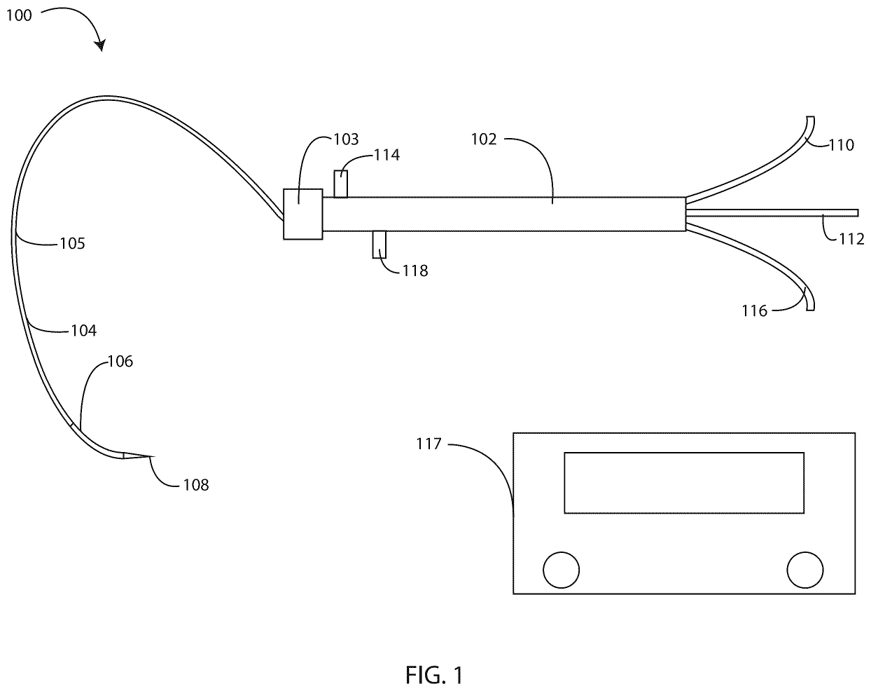

Referring now to FIG. 1, a schematic view of a cryoablation system is shown in accordance with various embodiments herein. In various embodiments, the cryoablation system can include a handle 102 and a shaft 104. In various embodiments, the shaft 104 is insertable into the handle 102 and can be securely attached to the handle with shaft-handle connector 103. In various embodiments, the shaft 104 and the shaft-handle connector 103 of a cryoablation system 100 can form a catheter assembly. In some embodiments, the catheter assembly includes the components of the cryoablation system that are to be replaced each time a cryoablation procedure is performed. In some aspects, the cryoablation system 100 may include a working fluid source 110, a pre-cooler fluid source 112, and vacuum source 114 which are connectible to the cryoablation system 100.

The three sources correspond to three independent circuits in the cryoablation system 100: pre-cooler, working fluid, and active vacuum. In some embodiments, the working fluid source 110 and pre-cooler fluid source 112 connect to the base of the handle 102 of the cryoablation system 100 and vacuum source 114 connects near the distal end of the handle, adjacent to the shaft-handle connector 103. The cryoablation system may further include a pre-cooler gas exhaust 116 and a working gas exhaust 118 connecting to the handle 102. In various embodiments, the shaft-handle connector 103 functions as a manifold to ensure each of the flow circuits remain isolated from one another.

In some embodiments, the cryoablation system 100 includes a console 117. The console may be used to control the system and may be in electrical and fluid communication with the handle and cryoablation assembly. In some embodiments, the working fluid source 110, pre-cooler fluid source 112, vacuum source 114 may all be connectable to a console 117 of the cryoablation system 100 using conduits. In some embodiments, the pre-cooler gas exhaust 116, working gas exhaust 118, or both can connect to a conduit which carries the exhaust back to the console 117 or other location in the procedure room where the exhaust is vented to the ambient environment at an appropriate location. It should be noted that various sources and exhausts may be placed in position and in any suitable configuration along the handle 102, and that the arrangement of FIG. 1 is just one example of a suitable configuration.

An example of specifications and functions of each of these circuits is provided in the following paragraph. However, it should be noted that the particular fluids and pressure values are meant for exemplary purposes and other configurations are possible.

In an embodiment, the pre-cooler circuit can contain 24.1 mega Pascals (MPa) pressurized Argon. The precooler circuit can cool the incoming stream of working fluid and can operate in the handle. In an embodiment, the working fluid circuit can contain 12.4 MPa pressurized Argon and/or 12.4 MPa pressurized Helium. The working fluid circuit generates and/or thaws ice balls. The working fluid circuit can operate in the handle, the insulated portion or insulated zone of the shaft, and the expansion chamber of the shaft. In an embodiment, the active vacuum can hold a vacuum of less than or equal to 6.67 Pascals (Pa). The active vacuum can insulate the shaft. The active vacuum can operate in the handle and the insulated zone of the shaft.

In various embodiments, the working fluid circuit runs through both the handle 102 and the shaft 104 of the cryoablation system 100 and carries the fluid which both generates and thaws the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. The term “gas circuit” is also used throughout the application, and could be replaced with fluid circuit, liquid circuit, fluid chamber, gas chamber, or liquid chamber in various embodiments. The term “gas” is used throughout and could be replaced with fluid or liquid in various embodiments.

During the ablation (freeze cycle), 12.4 MPa argon is circulated through the probe to generate the ice ball in the patient's body surrounding the expansion chamber 106. The working fluid can be any suitable cooling fluid (e.g., nitrogen, air, argon, krypton, xenon, N2O, CO2, CF4). In some embodiments, the pressure of the high-pressure stream of the working fluid can be greater than or equal to 6.9 MPa, 8.3 MPa, 9.7 MPa, 11.0 MPa, 12.4 MPa, 17.2 MPa, 27.6 MPa, or 41.4 MPa. In some embodiments, the pressure of the high-pressure stream of the working fluid can be less than or equal to 55.2 MPa, 34.5 MPa, 20.7 MPa, 18.6 MPa, 16.5 MPa, 14.5 MPa, or 12.4 MPa. In some embodiments, the pressure of the high-pressure stream of the working fluid can fall within a range of 6.9 MPa to 41.4 MPa, or 8.3 MPa to 27.6 MPa, or 9.7 MPa to 16.5 MPa, or 11.0 MPa to 14.5 MPa, or can be about 12.4 MPa. Accordingly, in the embodiments where the working fluid is a cooling fluid, the temperature of the working fluid at the expansion chamber 106 can be about 190 Kelvin. In some embodiments, the temperature of the working fluid can be less than or equal to 250 Kelvin, 200 Kelvin, 150 Kelvin, or 100 Kelvin, or can be an amount falling within a range between any of the foregoing.

In various embodiments, the pre-cooler circuit is fully contained within the handle 102. In various embodiments, the pre-cooler circuit is located in a console 117 of the system. In various embodiments, the pre-cooler circuit is located in a part of the catheter just proximal to the handle. In various embodiments, the pre-cooler circuit is located in a part of the catheter just distal to the handle. The pre-cooler circuit operates using argon or any other suitable cooling fluid in various embodiments. In some embodiments, the high-pressure stream of the pre-cooler fluid may be at a pressure greater than the pressure of the high-pressure stream of the working fluid. The pre-cooler fluid may, for instance, be supplied at pressures greater than about 13.8 MPa. In some embodiments, the pressure of the pre-cooler fluid can be greater than or equal to 10.3 MPa, 13.8 MPa, 17.2 MPa, 20.7 MPa, or 24.1 MPa. In some embodiments, the pressure of the pre-cooler fluid can be less than or equal to 31.0 MPa, 29.3 MPa, 25.9 MPa, or 24.1 MPa. In some embodiments, the pressure of the pre-cooler fluid can fall within a range of 10.3 MPa to 31.0 MPa, or 13.8 MPa to 29.3 MPa, or 17.2 MPa to 27.6 MPa, or 20.7 MPa to 25.9 MPa, or can be about 24.1 MPa.

In some embodiments, the outer surface of the shaft 104 may be thermally insulated from the inner surface of the shaft. In various embodiments, the vacuum circuit or vacuum chamber runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft. In alternative embodiments, shaft insulation can be obtained by containing a non-circulating fluid or gas within an insulating shaft.

The shaft 104 can be of any suitable length capable of reaching the target anatomy in the subject. In some embodiments, the shaft length can be greater than or equal to 20 cm, 38 cm, 55 cm, 72 cm, or 90 cm. In some embodiments, the shaft length can be less than or equal to 150 cm, 135 cm, 120 cm, 105 cm, or 90 cm. In some embodiments, the shaft length can fall within a range of 20 cm to 150 cm, or 38 cm to 135 cm, or 55 cm to 120 cm, or 72 cm to 105 cm, or can be about 90 cm.

In various embodiments, certain portions of the shaft 104 may be flexible. In an embodiment, the entire length of the shaft may be flexible. For instance, the shaft may be bendable about its lengthwise axis. In some such embodiments, the shaft may have a shaft diameter configured such that the shaft may be sufficiently flexible to form a curve having a desired radius of curvature. For instance, the shaft may be sufficiently flexible, such that the shaft may form a curve having the smallest radius of curvature of less than or equal to 30 mm, 20 mm, 10 mm, or 5 mm.

In various embodiments, shaft 104 may include an insulated zone 105 and an expansion chamber 106. The insulated zone 105 defines the portion of shaft 104 that is insulated by the vacuum chamber. The expansion chamber 106 defines the portion of the shaft 104 that is not insulated by the vacuum and where the ice ball is generated. In various embodiments, flexible shaft carries high pressure working fluid from the handle 102 to the expansion chamber 106, where it undergoes a Joule-Thompson expansion and corresponding temperature change. The working fluid exits down the flexible shaft, through the handle, before venting to the atmosphere from the console, or into the handle and venting from the handle.

The distal end of the shaft may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

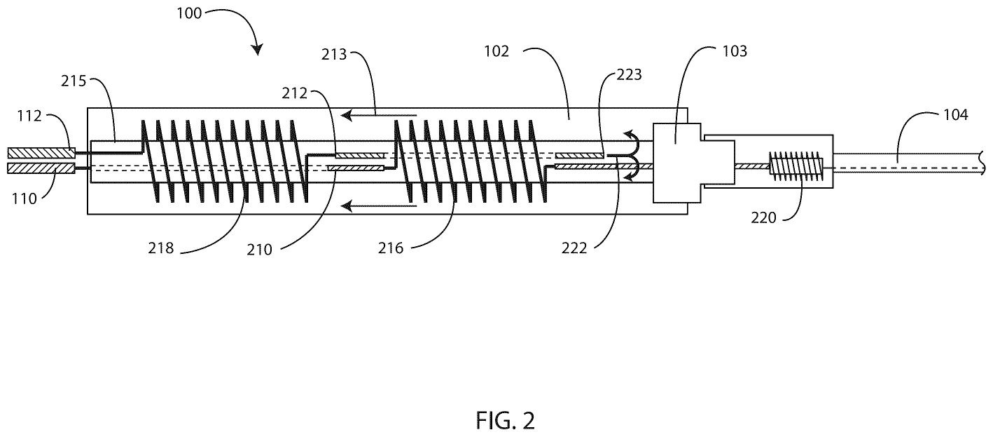

Handle with Pre-Cooler Circuit (FIG. 2)

Referring now to FIG. 2, a schematic view of portions of a cryoablation system is shown in accordance with various embodiments herein. In some aspects, the cryoablation system 100 may include a working fluid source 110 connecting to a working fluid circuit and a pre-cooler fluid source 112 connecting to a pre-cooler fluid circuit. The working fluid circuit may include a working fluid supply conduit 210 for carrying a high-pressure stream of the working fluid from the working fluid source 110 to the distal end of the shaft 104 (not shown in this view). The working fluid circuit may also include a working fluid return conduit (not shown in this view) for carrying a low-pressure stream of the working fluid from the distal end of the shaft back to the base of the handle 102.

The pre-cooler fluid circuit may include a pre-cooler supply circuit 212, which terminates at pre-cooler Joule-Thomson orifice 223 and carries a high-pressure stream of a pre-cooler fluid from the pre-cooler fluid source 112 to the pre-cooler fluid expansion region 222 in the handle 102. The pre-cooler fluid circuit may also include a pre-cooler return conduit (marked by arrows 213). The pre-cooler return conduit may be configured to carry the pre-cooler fluid away from the pre-cooler fluid expansion region 222 back to the base of the handle 102. The pre-cooler return conduit may be housed along with the pre-cooler supply circuit 212 and extend back to a control console and gas manifold.

In various embodiments, the pre-cooler fluid circuit may facilitate heat exchange between the working fluid and the pre-cooler fluid. For instance, the pre-cooler fluid circuit can be used to precool the high-pressure stream of the working fluid in embodiments where the working fluid cools upon expansion to cryogenically ablate tissue surrounding the distal operating tip 108. In various embodiments, the working fluid supply conduit 210 may include a first heat exchanger 216. The first heat exchanger 216 may facilitate heat exchange between the high-pressure stream of the working fluid in the working fluid supply conduit 210 and the low-pressure stream of the pre-cooler fluid in the pre-cooler return conduit.

In various embodiments, the pre-cooler supply conduit 212 may include a second heat exchanger 218 that permits heat exchange between the high-pressure stream of the pre-cooler fluid and the low-pressure stream of the pre-cooler fluid (e.g., recuperative heat exchange). In various embodiments aspects, the pre-cooler fluid may also be a cooling fluid. In such embodiments, recuperative heat exchange between the high-pressure stream of the pre-cooler fluid and the low-pressure stream of the pre-cooler fluid may remove heat from the high-pressure stream of the pre-cooler fluid. Accordingly, the second heat exchanger 218 may facilitate precooling the high-pressure stream of the pre-cooler fluid.

In various embodiments, the high-pressure stream of the pre-cooler fluid leaving the second heat exchanger 218 continues to flow through the pre-cooler supply conduit 212 to the pre-cooler fluid expansion region 222. In the pre-cooler fluid expansion region, which is fully contained in handle 102, the pre-cooler supply conduit 212 terminates in a Joule-Thomson orifice. The high-pressure stream of the pre-cooler fluid may undergo expansion at or downstream of the Joule-Thomson orifice in the pre-cooler fluid expansion region 222. The rapid drop in pressure causes a corresponding drop in temperature. The pre-cooler fluid expansion region 222 may be in fluid communication with the pre-cooler return conduit to carry the expanded low-pressure stream of the pre-cooler fluid (e.g., to vent to atmosphere, if the pre-cooler fluid circuit is an open circuit, or back to a pre-cooler fluid source if the pre-cooler fluid circuit is a closed circuit). After expansion at the Joule-Thomson orifice, the chilled pre-cooler fluid passes back through handle 102, in the annular space between the core tube 215 and the outer surface of the handle 102. As the pre-cooler fluid passes through the pre-cooler return conduit, it cools the working fluid at the first heat exchanger 216.

The working fluid circuit 210 may also include a third heat exchanger 220 in the shaft 104 of the cryoablation system that is configured for heat exchange (e.g., recuperative heat exchange) between the high-pressure stream of the working fluid in the working fluid supply circuit 210 and the low-pressure stream of the working fluid returning through the shaft 104 (not shown in this view).

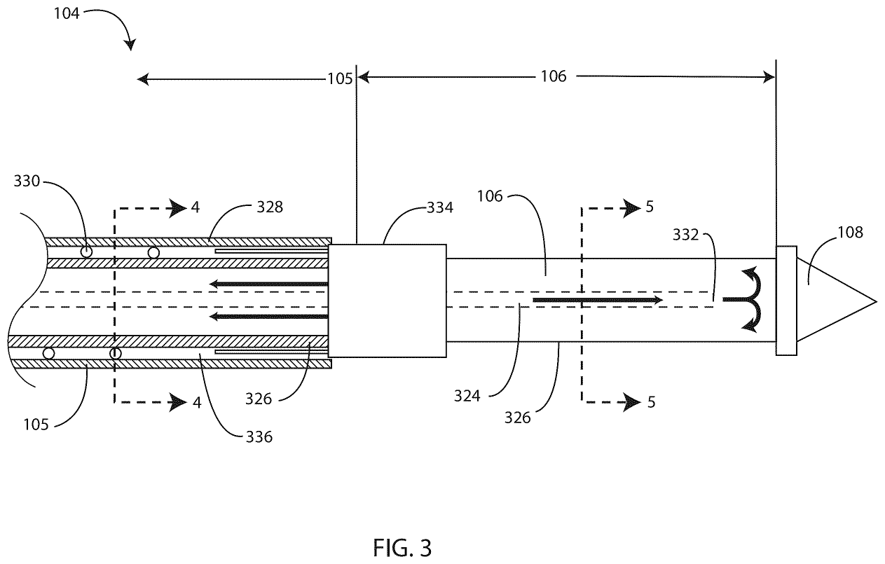

### Distal Tip and Expansion Chamber Details (FIG. 3)

Referring now to FIG. 3, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 which is located within a return tube 326, which is located within an insulating shaft 328. The concentric-shaft construction is designed to isolate the working fluid circuit 210 and vacuum chamber 336 from each other.

In various embodiments, after exiting the handle 102, the high-pressure flow of the working fluid travels down the supply tube 324. When the working fluid reaches the working fluid expansion chamber 106, the supply tube 324 terminates in a Joule-Thomson orifice 332 or distal outlet 332. The high-pressure stream of the working fluid may undergo expansion at or downstream of the Joule-Thomson orifice 332 in expansion chamber 106. The rapid drop in pressure causes a corresponding drop in temperature. Heat transfer between the expanded working and the outer walls of expansion chamber 106 leads to formation of an ice ball in the tissue around the tip 108 resulting in cryoablation of the tissue.

The expansion chamber 106 may be in fluid communication with the working fluid return conduit (defined by the annular space between the supply tube 324 and the inner surface of the return tube 326 of the expansion chamber) to carry the expanded low pressure stream of the working fluid (e.g., to vent to atmosphere, if the working fluid circuit is an open circuit, or back to a working fluid source if the working fluid circuit is a closed circuit). As the working fluid passes through the working fluid return conduit, it cools the working fluid input stream at the third heat exchanger 220 (FIG. 2).

In various embodiments, the working fluid is a cooling fluid and a cooling gas (e.g., nitrogen, air, argon, krypton, xenon, N₂O, CO₂, CF₄). In such cases, the high-pressure stream of the working fluid may be at a pressure such that expansion via the Joule-Thomson orifice 332 may result in the working fluid cooling to temperatures for cryogenically ablating tissue surrounding the expansion chamber 106. In certain aspects, the pressure of the high-pressure stream of the working fluid upstream of the Joule-Thomson orifice 332 can be between about 6.9 MPa and about 13.8 MPa (e.g., about 12.4 MPa). Accordingly, in the embodiments where the working fluid is a cooling fluid, the temperature of the working fluid after expansion from the Joule-Thomson orifice 332 can be greater than or equal to 150, 160, 170, 180, 190, or 200 Kelvin, or can be an amount falling within a range between any of the foregoing.

Cryoablation system 100 can be designed such that the outermost surface of the shaft does not cause thermal damage to non-target structures. In various embodiments, Ice ball formation is limited to the expansion chamber 106 of the shaft 104, which can also be referred to as the active region of the device. Selective ice ball formation is achieved by pulling a vacuum through the insulated zone 105 of shaft 104. In various embodiments, the cryoablation system 100 may be configured for establishing vacuum communication between the shaft 104 and vacuum source 114.

Referring to FIG. 1, cryoablation system 100 may be configured to connect to a vacuum source 114 at handle 102. In various embodiments, the vacuum source 114 is configured to pull vacuum along the length of the insulated zone 105 of shaft 104. In an embodiment, vacuum is pulled between the outer diameter of the return tube 326 and the inner diameter of the insulating shaft 328 throughout the insulated zone 105 of shaft 104.

In various embodiments, the vacuum source 114 is configured to pull a vacuum within at least a portion of the handle 102. Such a configuration can insulate the handle 102 and protect the cryoablation system operator from cryogenic exhaust gases. In some embodiments the vacuum source 114 is connected to the handle 102 and the shaft 104 is in fluid communication with the handle 102 such that pulling a vacuum in the handle can also evacuate the space between the supply tube 324 and return tube 326. In other embodiments, the vacuum source 114 is connected directly to the shaft 104, for example, with the use of a T-fitting along the length of the shaft 104.

To provide for thermal insulation along the insulated zone 105 of shaft 104, the wall of the flexible shaft is a double wall (a return tube surrounded by an insulating shaft) with a small gap between the return tube 326 and the insulating shaft 328. By pulling a vacuum between the return tube and the insulating shaft, convective heat transfer is prevented, so that the temperature of the working fluid does not ablate or cause uncontrolled apoptosis/necrosis to healthy non-target patient tissue along the insulated zone of the shaft. Adequate thermal insulation is obtained by actively pumping out the air in the gap and maintaining a vacuum of about 0.05 torr. However, other vacuum pressures may be appropriate depending on the configuration of the cryoablation system. In some embodiments, a supporting filament 330 is wrapped around the outer diameter of the return tube 326. One option for the filament material is a polymer such as polyether ether ketone (PEEK). The filament may prevent direct contact between the return tube outer surface and insulating shaft inner surface. Filament 330 minimizes thermal conduction between the inner and insulating shafts. Other alternatives may be used in place of the filament 330 such as an extruded tubing/co-extrusion shape or other features placed onto the shaft.

In some embodiments, the shaft may not include a filament. In such embodiments the return tube 326 and the insulating shaft 328 are selected to have material properties that are sufficient to minimize thermal conduction between the inner shaft and the insulating shaft.

A joint 334 is present at the junction of the insulated zone 105 and the expansion chamber 106. This joint is capable of sealing the vacuum layer.

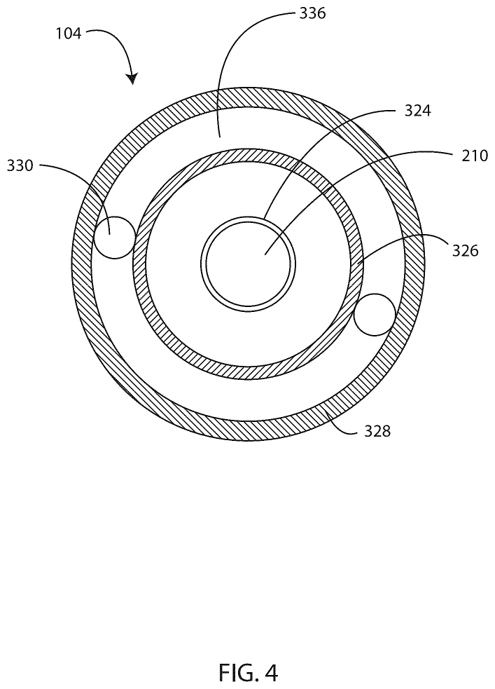

### Flexible Shaft Cross-Section, Dimensions and Materials (FIG. 4)

Referring now to FIG. 4, a cross sectional view of the shaft of FIG. 3 taken along section 4-4 is shown in accordance with various embodiments herein. In various embodiments, the insulated zone 105 of shaft 104 includes a supply tube 324 concentrically located within a return tube 326, which is concentrically located within an insulating shaft 328. The insulated zone 105 can include a shaft portion of the vacuum chamber 336 and an insulated portion of the working gas circuit 210. In various embodiments, the vacuum chamber 336 surrounds and is isolated from the insulated portion of the working gas circuit 210.

In various embodiments, after exiting the handle 102, the high-pressure flow of the working fluid travels distally down the insulated zone of the shaft through supply tube 324. After cooling and expansion in the expansion chamber 106, the working fluid travels proximally back through the insulated zone 105 of the shaft 104 in the annular space between the supply tube 324 and return tube 326.

In various embodiments, the materials and dimensions of each of the layers of the shaft 104 may be selected to provide sufficient degree of flexibility for the shaft to be bendable about its longitudinal axis at the working temperatures of the device.

In various embodiments, the supply tube 324, which also may be referred to as a capillary tube herein, is constructed from any suitable material or materials such as flexible metals, polymers, composites, or the like. In an embodiment, the supply tube 324 is constructed from Nitinol (NiTi), stainless steel, or the like.

In some embodiments, the inner diameter of the supply tube 324 can be greater than or equal to 0.30 mm, 0.35 mm, 0.40 mm, or 0.45 mm. In some embodiments, the inner diameter of the supply tube 324 can be less than or equal to 0.60 mm, 0.55 mm, 0.50 mm, or 0.45 mm. In some embodiments, the diameter of the supply tube 324 can fall within a range of 0.30 mm to 0.60 mm, or 0.35 mm to 0.55 mm, or 0.40 mm to 0.50 mm, or can be about 0.45 mm.

In some embodiments, the outer diameter of the supply tube 324 can be greater than or equal to 0.38 mm, 0.43 mm, 0.48 mm, 0.53 mm, or 0.58 mm. In some embodiments, the outer diameter can be less than or equal to 0.78 mm, 0.73 mm, 0.68 mm, 0.63 mm, or 0.58 mm. In some embodiments, the outer diameter can fall within a range of 0.38 mm to 0.78 mm, or 0.43 mm to 0.73 mm, or 0.48 mm to 0.68 mm, or 0.53 mm to 0.63 mm, or can be about 0.58 mm.

In some embodiments, the thickness of the supply tube 324 can be greater than or equal to 0.10 mm, 0.11 mm, 0.12 mm, 0.14 mm, or 0.15 mm. In some embodiments, the thickness of the supply tube 324 can be less than or equal to 0.20 mm, 0.19 mm, 0.18 mm, 0.16 mm, or 0.15 mm. In some embodiments, the thickness of the supply tube 324 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the return tube 326 is constructed from any suitable material or materials such as flexible metals, polymers, or the like. In various embodiments, the return tube 326 can be made of polyimide, fluorinated ethylene propylene (FEP), Teflon, or the like. In an embodiment, the return tube 326 is formed from a polyimide material as it is highly impermeable to gases at a wide range of temperatures and can thus contain the working fluid inside and hold vacuum on the outside. In a particular example, the return tube 326 is made of a braid-reinforced polyimide tube to enhance gas impermeability, burst strength, and flexibility. In some embodiments, the return tube 326 is formed from a single layer of material. In some embodiments, the return tube 326 can be formed from two or more layers of material selected to optimize the performance of the shaft 104. The layers of material can be bonded together using any suitable technique or techniques such as adhesives, reflow processes, or the like.

In some embodiments, the outer diameter of the return tube 326 can be greater than or equal to 1 mm, 1.1 mm, 1.2 mm, 1.3 mm, or 1.4 mm. In some embodiments, the outer diameter of the return tube 326 can be less than or equal to 1.8 mm, 1.7 mm, 1.6 mm, 1.5 mm, or 1.4 mm. In some embodiments, the outer diameter of the return tube 326 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the inner diameter of the return tube 326 can be greater than or equal to 0.9 mm, 1.0 mm, 1.1 mm, 1.2 mm, or 1.3 mm. In some embodiments, the inner diameter of the return tube 326 can be less than or equal to 1.7 mm, 1.6 mm, 1.5 mm, 1.4 mm, or 1.3 mm. In some embodiments, the inner diameter of the return tube 326 can fall within a range of 0.9 mm to 1.7 mm, or 1.0 mm to 1.6 mm, or 1.1 mm to 1.5 mm, or 1.2 mm to 1.4 mm, or can be about 1.3 mm.

In some embodiments, the thickness of the return tube 326 can be greater than or equal to 0.10 mm, 0.11 mm, 0.12 mm, 0.14 mm, or 0.15 mm. In some embodiments, the thickness of the return tube 326 can be less than or equal to 0.20 mm, 0.19 mm, 0.18 mm, 0.16 mm, or 0.15 mm. In some embodiments, the thickness of the return tube 326 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the insulating shaft 328 is constructed from any suitable material or materials such as flexible metals, polymers, or the like. In various embodiments, the insulating shaft 328 is made of polyimide, fluorinated ethylene propylene (FEP), Teflon, or the like. In a particular embodiment, the insulating shaft 328 may include polytetrafluoroethylene (PTFE), and/or one or more polyether block amides (known under the tradename Pebax®, hereinafter “Pebax”).

In some embodiments, the insulating shaft 328 is formed from a single layer of material. In some embodiments, the insulating shaft 328 can be formed from two or more layers of material selected to optimize the performance of the shaft 104. The layers of material can be bonded together using any suitable technique or techniques such as adhesives, reflow processes, or the like.

In an embodiment, the insulating shaft may be formed using a braid-reinforced polyimide tube skim-coated with a Pebax outer layer. Such a tri-layer construction enables the deep vacuum to be maintained between the return tube and the insulating shaft without causing the insulating shaft 328 to collapse onto the return tube 326.

In some embodiments, the outer diameter of the insulating shaft 328 can be greater than or equal to 1.2 mm, 1.4 mm, 1.5 mm, 1.6 mm, or 1.8 mm. In some embodiments, the outer diameter of the insulating shaft can be less than or equal to 2.2 mm, 2.1 mm, 2.0 mm, 1.9 mm, or 1.8 mm. In some embodiments, the outer diameter of the insulating shaft can fall within a range of 1.3 mm to 2.3 mm, or 1.4 mm to 2.1 mm, or 1.5 mm to 2.0 mm, or 1.6 mm to 1.9 mm, or can be about 1.8 mm.

In some embodiments, the inner diameter of the insulating shaft 328 can be greater than or equal to 1.0 mm, 1.2 mm, 1.3 mm, 1.4 mm, or 1.6 mm. In some embodiments, the inner diameter of the insulating shaft 328 can be less than or equal to 2.2 mm, 2.0 mm, 1.9 mm, 1.8 mm, or 1.6 mm. In some embodiments, the inner diameter of the insulating shaft 328 can fall within a range of 1.0 mm to 2.2 mm, or 1.2 mm to 2.0 mm, or 1.3 mm to 1.9 mm, or 1.4 mm to 1.8 mm, or can be about 1.6 mm.

In some embodiments, the thickness of the insulating shaft 328 can be greater than or equal to 0.10 mm, 0.11 mm, 0.12 mm, 0.14 mm, or 0.15 mm. In some embodiments, the thickness of the insulating shaft 328 can be less than or equal to 0.20 mm, 0.19 mm, 0.18 mm, 0.16 mm, or 0.15 mm. In some embodiments, the thickness of the insulating shaft 328 can fall within a range of 0.10 mm to 0.20 mm, or 0.11 mm to 0.19 mm, or 0.12 mm to 0.18 mm, or 0.14 mm to 0.16 mm, or can be about 0.15 mm.

In some embodiments, a PEEK filament 330 is wound around the return tube 326. PEEK filament 330 may have pitch that is greater than or equal to 0.5 mm, 1.0 mm, 1.5 mm, or 2.0 mm, or can be an amount falling within a range between any of the foregoing. Alternatively, the filament may be a plurality of discrete pieces attached along the return tube 326. The PEEK filament 330 prevents direct contact between the return tube 326 and the insulating shaft 328, maintaining their coaxial alignment. In some embodiments, an adhesive (e.g., Loctite) is applied on the filament at the end of the return tube 326 and the insulating shaft 328 to attach the PEEK filament 330. In various embodiments, the PEEK filament wrap is configured to minimize or prevent conductive heat transfer from the return tube to the insulating shaft. In alternative embodiments, other insulating polymers may be used as a substitute for the PEEK filament such as expanded PTFE (ePTFE), nylon, or the like.

In some embodiments, the diameter of the PEEK filament 330 can be greater than or equal to 0.002 mm, 0.004 mm, or 0.005 mm. In some embodiments, the diameter of the PEEK filament 330 can be less than or equal to 0.007 mm, 0.006 mm, or 0.005 mm. In some embodiments, the diameter of the PEEK filament 330 can fall within a range of 0.002 mm to 0.007 mm, or 0.004 mm to 0.006 mm, or can be about 0.005 mm.

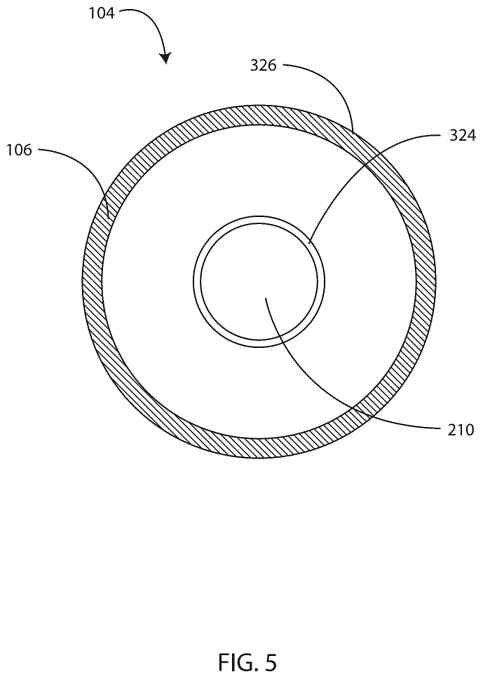

### Shaft in the Expansion Chamber (FIG. 5)

Referring now to FIG. 5, a cross-sectional view of the shaft of FIG. 3 taken along section 5-5 is shown in accordance with various embodiments herein. The cross-sectional view of FIG. 5 depicts expansion chamber 106 of the shaft 104. In various embodiments, the expansion chamber 106 is distal to the insulated zone 105 along the shaft 104. The expansion chamber 106 can include an expansion portion of the working fluid circuit 210.

In various embodiments, after exiting the handle 102, the high-pressure flow of the working fluid travels down the supply tube 324. After cooling and expansion in expansion chamber 106, the working fluid travels back down through the expansion chamber 106 in the annular space between the supply tube 324 and the outer wall of the expansion chamber. In various embodiments, the expansion chamber 106 is configured to maximize the heat transfer between the working gas and the patient's tissue through the optimization of parameters such as wall thickness, materials, and the like.

In various embodiments, the expansion chamber 106 is constructed from any suitable material or materials such as flexible metals, polymers, or the like. In various embodiments, the expansion chamber 106 is made of polyimide, fluorinated ethylene propylene (FEP), Teflon, or the like. In some embodiments, the expansion chamber 106 includes a continuation of the return tube 326 of the insulated zone 105 of the shaft 104. Alternatively, the expansion chamber is a separate component from the return tube 326 that can be joined to the shaft 104 using any suitable joint and/or fitting, such as reflow processes, glue joints, solder joints, or any other suitable mechanical joining process capable of withstanding cryogenic pressures and temperatures.

In some embodiments, the expansion chamber 106 is formed from a single layer of material. In some embodiments, the expansion chamber 106 is formed from two or more layers of material. The layers of material can be bonded together using any suitable technique or techniques such as adhesives, reflow processes, or the like.

In some embodiments, the outer diameter of the expansion chamber 106 can be greater than or equal to 1.3 mm, 1.4 mm, 1.5 mm, 1.6 mm, or 1.7 mm. In some embodiments, the outer diameter of the expansion chamber 106 can be less than or equal to 2.1 mm, 2.0 mm, 1.9 mm, 1.8 mm, or 1.7 mm. In some embodiments, the outer diameter of the expansion chamber 106 can fall within a range of 1.3 mm to 2.1 mm, or 1.4 mm to 2.0 mm, or 1.5 mm to 1.9 mm, or 1.6 mm to 1.8 mm, or can be about 1.7 mm.

In some embodiments, the inner diameter of the expansion chamber 106 can be greater than or equal to 1.0 mm, 1.1 mm, 1.2 mm, 1.3 mm, or 1.4 mm. In some embodiments, the inner diameter of the expansion chamber 106 can be less than or equal to 1.8 mm, 1.7 mm, 1.6 mm, 1.5 mm, or 1.4 mm. In some embodiments, the inner diameter of the expansion chamber 106 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the thickness of the wall of expansion chamber 106 can be greater than or equal to 0.20 mm, 0.22 mm, 0.25 mm, 0.28 mm, or 0.30 mm. In some embodiments, the thickness of the wall of the expansion chamber 106 can be less than or equal to 0.40 mm, 0.38 mm, 0.35 mm, 0.32 mm, or 0.30 mm. In some embodiments, the thickness of the wall of the expansion chamber 106 can fall within a range of 0.20 mm to 0.40 mm, or 0.22 mm to 0.38 mm, or 0.25 mm to 0.35 mm, or 0.28 mm to 0.32 mm, or can be about 0.30 mm.

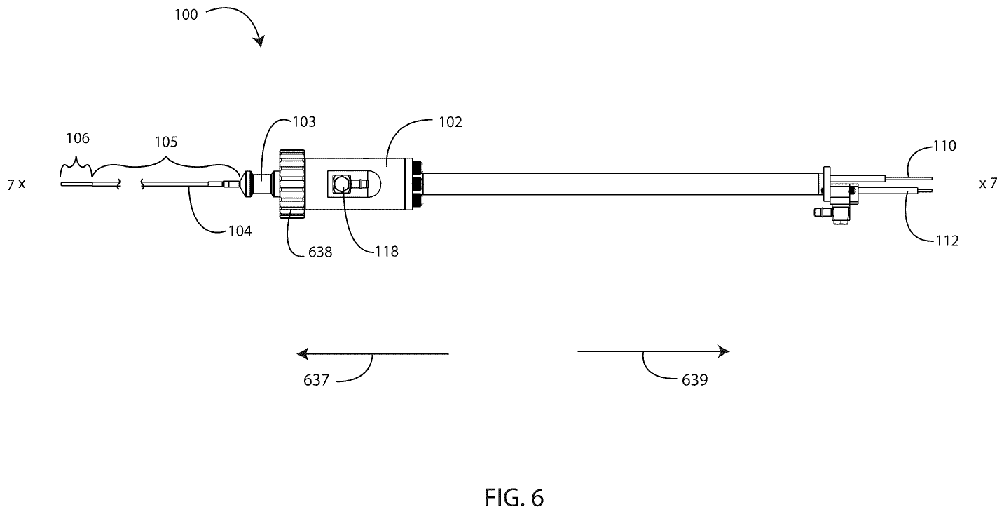

### Cryoablation System (FIGS. 6-8)

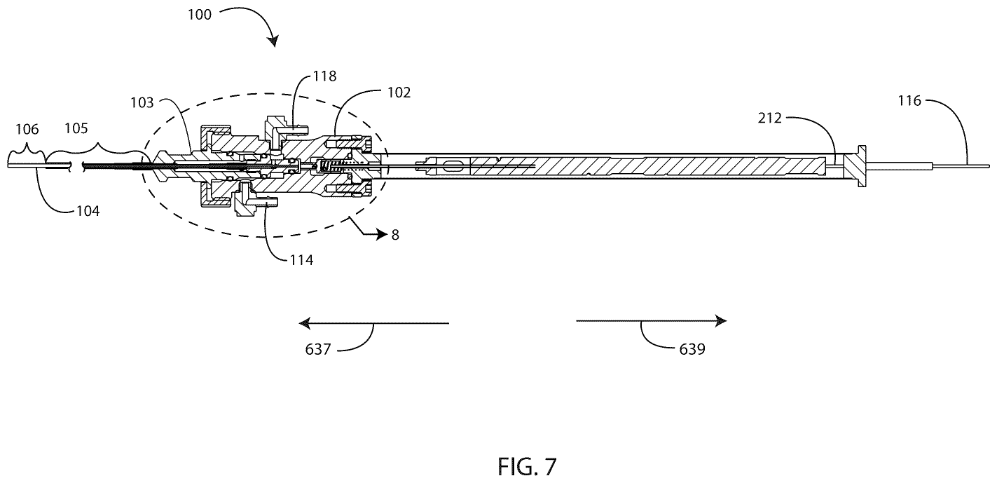

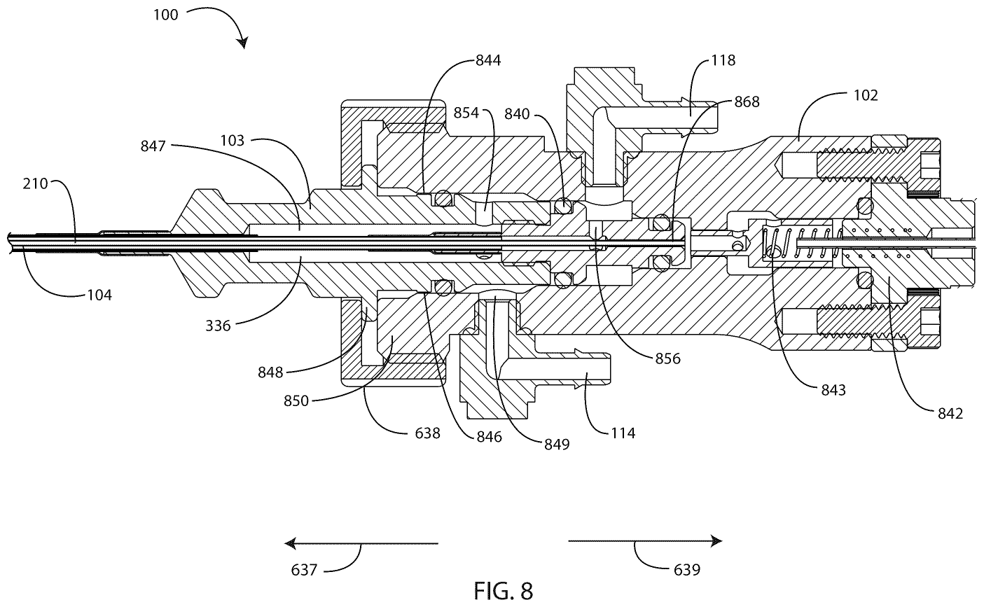

Referring now to FIG. 6-8, various views of a cryoablation system are shown herein. FIG. 6 is a schematic side view of a cryoablation system in accordance with various embodiments herein. FIG. 7 is a cross-sectional view of the cryoablation system of FIG. 6 along line 7-7 of FIG. 6, looking into the plane of the page, in accordance with various embodiments herein. FIG. 8 is a closeup view of the cryoablation system of FIG. 7 about detail 8 of FIG. 7 in accordance with various embodiments herein. In reference to the FIGS., arrows have been added to denote distal direction 637 and proximal direction 639.

In various embodiments, the cryoablation system 100 can include a handle 102 and a shaft 104. In some aspects, the cryoablation system 100 may include a working gas source 110, a pre-cooler gas source 112, and vacuum source 114, which are connectible to the cryoablation system 100. The three sources correspond to three independent circuits in the cryoablation system 100: pre-cooler gas supply circuit 212, working gas circuit 210, and vacuum chamber 336. In the embodiment of FIGS. 6-8, the working gas source 110 and pre-cooler gas source 112 connect to the cryoablation system 100 at the proximal end of the handle 102 and the vacuum source 114 connects to the cryoablation system near the distal end of the handle. However, the three sources may be connected along any suitable portion of the handle 102.

In addition, or alternatively, the cryoablation system may include two, three, four, or more pre-cooler gas sources. Alternatively, the cryoablation system 100 does not include a separate pre-cooler gas source. In such embodiments, the cryoablation system may have a multistage cooling system in which the pressure of the working gas is stepped down in multiple stages, such as two, three, or four stages. For example, the working gas pressure may be stepped down in two stages, such as from about 4000 psi to about 2000 psi in a first stage and from about 2000 psi to about 500 psi in a second stage.

In various embodiments, the pre-cooler gas supply circuit 212 passes through and is present within the handle 102. When referring to the portions of the pre-cooler gas supply circuit 212 that pass through the handle 102, the term “handle portion of the pre-cooler gas supply circuit” will be used. Similarly, the vacuum chamber 336 and working gas circuit 210 have portions that pass through the handle 102, and these will be referred to as the “handle portion of the vacuum chamber” and the “handle portion of the working gas circuit”, respectively, herein.

In various embodiments, the handle 102 can include a handle portion of the pre-cooler gas supply circuit 212, a handle portion of the vacuum chamber 336, and a handle portion of the working gas circuit 210. In various embodiments, the pre-cooler gas supply circuit 212 is configured to supply a pre-cooler gas from a high-pressure cryogenic gas source (in this case pre-cooler gas source 112) to the handle 102. As shown and described in FIG. 2, the pre-cooler gas circuit can include a pre-cooler Joule-Thomson orifice at pre-cooler gas expansion region 222.

The shaft 104 can include an insulated zone 105 along a proximal length of the shaft. The insulated zone 105 can include a shaft portion of the vacuum chamber 336 and an insulated portion of the working gas circuit 210. As best seen by FIG. 4, the vacuum chamber 336 surrounds and is isolated from the insulated portion of the working gas circuit 210. The shaft can include a working gas expansion chamber 106 distal to the insulated zone 105. As best seen by FIG. 5, the working gas expansion chamber 106 comprises an expansion portion of the working gas circuit 210.

As shown and described in FIGS. 2-3, the working gas circuit 210 is configured to supply a working gas from a high-pressure cryogenic gas source (in this case working gas source 110) to the working gas expansion chamber 106, the working gas circuit comprising a working gas Joule-Thomson orifice 332 at the working gas expansion chamber 106.

In various embodiments, shaft 104 is insertable into the handle 102 and can be securely attached to the handle with shaft-handle connector 103. A proximal end of the shaft 104 is configured to connect to the shaft-handle connector 103 and the shaft-handle connector is configured to removably attach the proximal end of the shaft 104 to a distal end of the handle 102. In the context of this application, when two components are removably attachable, a first component (e.g., a handle of a cryoablation system) can be attached to and/or detached from a second component (e.g., a shaft of a cryoablation system) without damaging the first component. In some examples, a first component can be attached to and/or detached from a second component without damaging either the first component or the second component. In other examples, a second component can be attached to and/or detached from a first component while intentionally plastically deforming specific components of the second component, and without damaging the first component.

In the context of the cryoablation system 100, the shaft-handle connector 103 allows for a shaft 104 to be removed from the handle 102 without damaging the handle. The shaft-handle connector 103 further allows the pre-cooler gas supply circuit 212, working gas circuit 210, and vacuum chamber 336 to remain isolated from one another within the handle 102 as the shaft 104 is removed from the handle. Such a configuration can improve the efficiency of a cryoablation system 100 because in many applications, the shaft 104 is replaced each time a cryoablation procedure is performed, but the handle 102 can be reused. The shaft-handle connector 103 enables a user of the cryoablation system 100 to remove a first shaft 104 from the handle 102 of the cryoablation system and replace it with a second shaft (not pictured).

In the example of FIGS. 6-8, the shaft 104 is permanently attached to the shaft-handle connector 103 and the shaft-handle connector 103 is removably attached to the handle 102 with a securing means (e.g., fastener 638). The shaft 104 can be removed from the handle 102 by releasing the securing means and removing the shaft-handle connector 103 from the handle 102. Additionally, or alternatively, the shaft-handle connector 103 can be removably connected to the shaft 104 making it possible for the shaft to be removed from the handle 102 without removing the shaft-handle connector 103.

In an alternate embodiment, the reusable handle 102 is configured to consume the shaft 104. For instance, the shaft 104 is connected to the handle 102 by clamping the handle onto the shaft (e.g., by using one or more Yor-Lok fittings, or the like). In such an embodiment, the handle 102 is bound onto the shaft 104 such that the shaft is consumed by the handle (e.g., portions of the shaft are plastically deformed, and the shaft is sacrificed), but the handle is not damaged and suitable for reuse. A Yor-Lok fitting is a compression fitting designed to handle higher pressure fluid connections. A body and nut are provided along with two ferrules or sleeves, which are positioned on the front and back of the body and nut, to make an air-tight seal.

In the embodiment of FIGS. 6-8, a handle inner surface 844 is configured to form a seal around a shaft-handle connector outer surface 846. In some embodiments, the shaft-handle connector 103 can include one or more O-rings 840 (or another sealing means) configured to enhance the seal between the handle 102 and the shaft-handle connector. Additionally, or alternatively, the handle 102 may include one or more 0-rings (or another sealing means) configured to enhance the seal between the handle 102 and the shaft-handle connector 103. Additionally, or alternatively, the handle 102 may include one or more metal or plastic components, in conjunction with one or more 0-rings (or another sealing means) configured to seal via plastic deformation of the materials between the handle 102 and the shaft-handle connector 103.

The cryoablation system 100 may further include a securing means to secure the shaft-handle connector 103 to the handle. In the example of FIGS. 6-8, the shaft-handle connector 103 includes a fastener 638. Fastener 638 is configured to rest on a connector protrusion 848 of the shaft-handle connector 103 and secure to a handle protrusion 850 of the handle 102. In an embodiment, the fastener 638 and the handle protrusion 850 may both be threaded and the fastener 638 can be screwed into the handle protrusion 850 to securely and removably attach the shaft-handle connector 103 to the handle 102. It should be noted that any other securing means configured to securely and removably attach the shaft-handle connector 103 to the handle 102 can be used.

As best seen in FIG. 8, the shaft-handle connector 103 includes a vacuum chamber connector portion 847. The shaft-handle connector 103 can define one or more vacuum openings 854 in fluid communication with the vacuum chamber connector portion 847. The vacuum openings 854 are configured to connect to a vacuum chamber handle portion 849, which is connected to vacuum source 114. In some embodiments, the vacuum chamber handle portion 849 runs along the length of the handle 102. In such an embodiment, the vacuum chamber provides a protective barrier between the handle 102 and an operator of the cryoablation system from the expanding pre-cooler gas in the handle. Alternatively to running along the length of the handle 102, the vacuum chamber handle portion 849 may terminate near the distal end of the handle.

In various embodiments, the vacuum chamber connector portion 847 is also in fluid communication with the vacuum chamber 336 running through the insulated zone 105 of the shaft 104. By fluidically connecting the vacuum source to the shaft 104 via the shaft-handle connector 103, a vacuum can be pulled along the length of the insulated zone 105 of the shaft 104 while remaining isolated from the pre-cooler gas supply circuit 212 and working gas circuit 210. In some embodiments, pulling a vacuum along the insulated zone 105 of the shaft 104 throughout the cryoablation procedure provides a protective barrier between the outer surface of the shaft 104 and the patient from the cryogenically cooled working gas. In some embodiments, a combination of an insulative material construction in combination with a vacuum pulled along the insulated zone 105 of the shaft 104 throughout the cryoablation procedure provides a further protective barrier between the outer surface of the shaft 104 and the patient from the cryogenically cooled working gas.

In some embodiments, the vacuum source 114 is an active vacuum. For instance, the vacuum source 114 may be a vacuum pump, or the like. The vacuum pump may be in operative communication with the vacuum chamber 336. The vacuum chamber is configured to prevent heat transfer by creating a low-pressure environment between the return tube 326 and the insulating shaft 328 in the insulated zone of the shaft. Alternatively, the vacuum source may be a passive vacuum, such as a vacuum sleeve or the like. Vacuum sleeves often consist of two layers of material, an inner layer and an outer layer, separated by a vacuum or low-pressure gap.

In various embodiments, the shaft-handle connector 103 defines one or more working gas openings 856 through which a return portion of the working gas circuit 210 runs between the handle 102 and the shaft-handle connector 103. The working gas openings 856 are configured to connect to the working gas exhaust 118. The working gas openings 856 are also in fluid communication with the insulated portion of the working gas circuit 210 such that after expansion in the expansion chamber 106 of the shaft 104 the working gas returns through the shaft and then exits through the working gas exhaust 118 via the working gas openings 856 in the shaft-handle connector 103. In some embodiments, the working gas exhaust 118 can connect to a conduit which carries the working gas exhaust back to the console or other location in the procedure room where it is vented to the ambient environment at an appropriate location. In another embodiment, the working gas exhaust 118 can connect to a conduit which carries the working gas exhaust through a chamber surrounding a conduit containing pre-cooler gas, so that the working gas exhaust cools the precooler gas to achieve increased thermal efficiency.

In various embodiments, the shaft-handle connector 103 can include a valve 842. In the example of FIGS. 6-8, valve 842 can be a check valve and include a spring 843. However, other suitable types of valves may be implemented. In various embodiments, the valve 842 can have an open state in which the working gas circuit 210 flows from the handle 102 to the shaft 104 through a conduit 868 of the shaft-handle connector 103 and a closed state in which the working gas circuit 210 cannot flow from the handle 102 to the shaft 104 through conduit 868 of the shaft-handle connector. In various embodiments, valve 842 is configured to switch from the open state to the closed state when a shaft 104 and shaft-handle connector 103 are removed from the handle 102. Such a feature is configured to prevent leakage of the working gas from the handle 102 when replacing a shaft 104. FIG. 8 shows the open state of the valve 842, where the spring 843 is depressed and therefore the valve is opened by the linear motion caused by threading the fastener 638 of the shaft-handle connector 103 onto the handle protrusion 850 of the handle 102. In various embodiments, the valve 842 is moved to an open state by the action of securing the shaft-handle connector 103 into a sealed engagement with the handle 102.

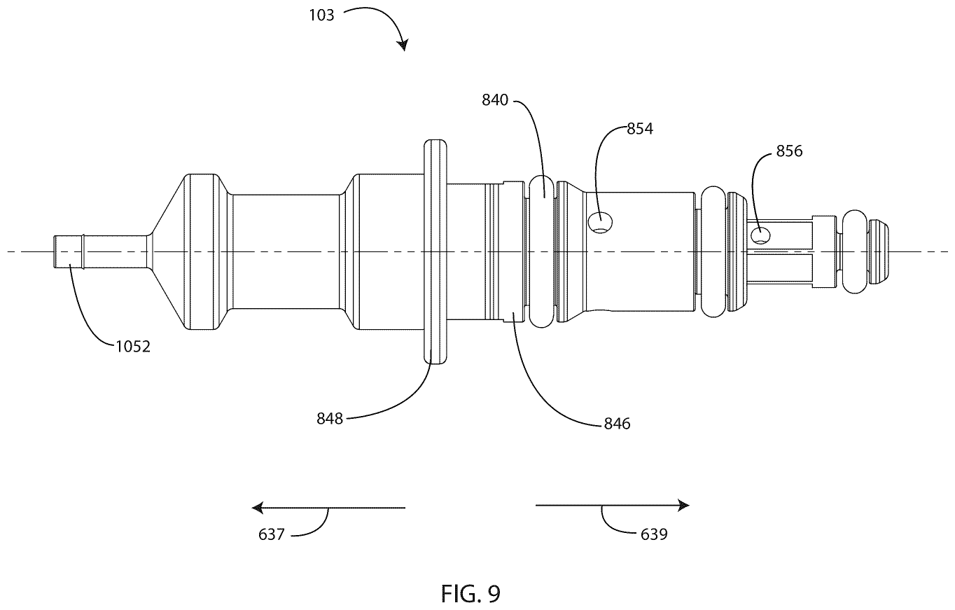

### Shaft-Handle Connector (FIGS. 9-11)

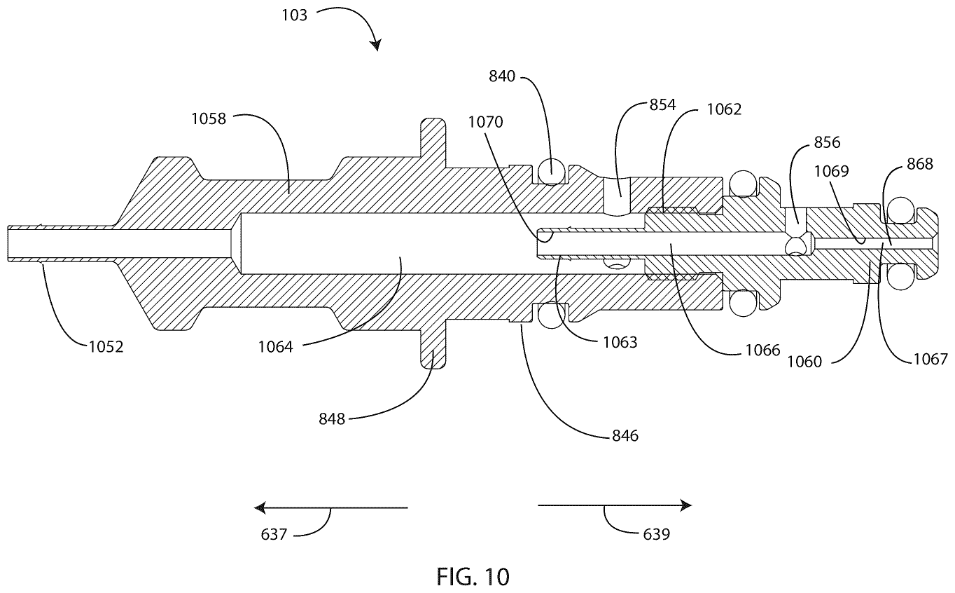

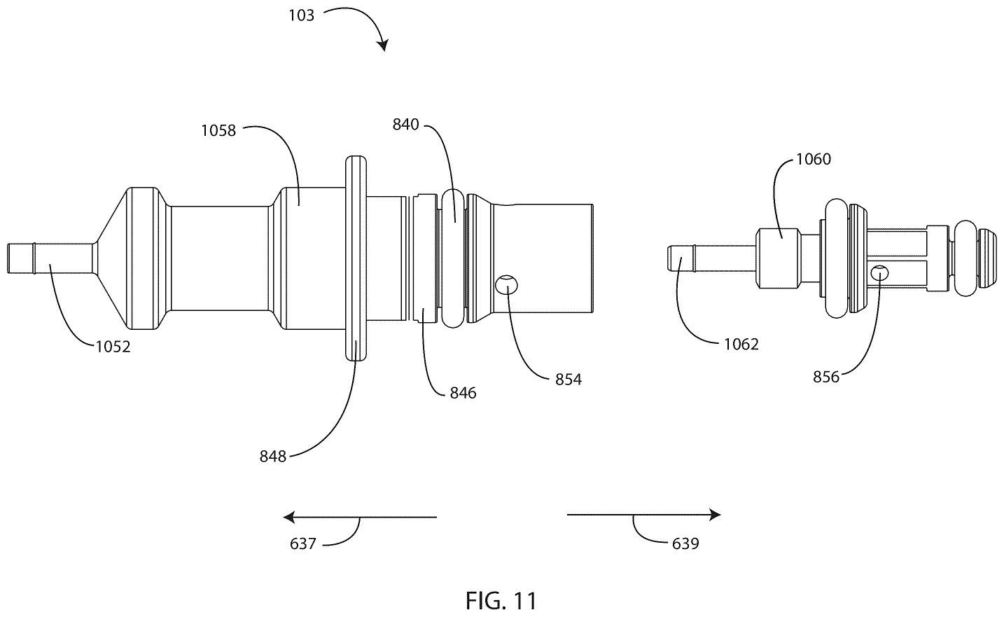

Referring now to FIG. 9-11, various views of a shaft-handle connector are shown herein. FIG. 9 is a schematic side view of a shaft-handle connector in accordance with various embodiments herein. FIG. 10 is a cross sectional view of the shaft-handle connector in accordance with various embodiments herein. FIG. 11 is an exploded view of the shaft-handle connector in accordance with various embodiments herein.

In various embodiments, the shaft-handle connector 103 can include a first connector piece 1058 and a second connector piece 1060. The first connector piece 1058 is configured to attach to the second connector piece 1060 by any suitable means. In some embodiments, the first connector piece 1058 is configured to permanently attach to the second connector piece 1060 by an interference fit, or the like. In some embodiments, the first connector piece 1058 is configured to removably attach to the second connector piece 1060. For instance, the first connector piece 1058 and a second connector piece 1060 can be threaded and the second connector piece 1060 can be screwed into the first connector piece 1058. Alternatively, the first connector piece 1058 and second connector piece 1060 may include one or more removable fittings to attach the first connector piece 1058 to the second connector piece 1060.

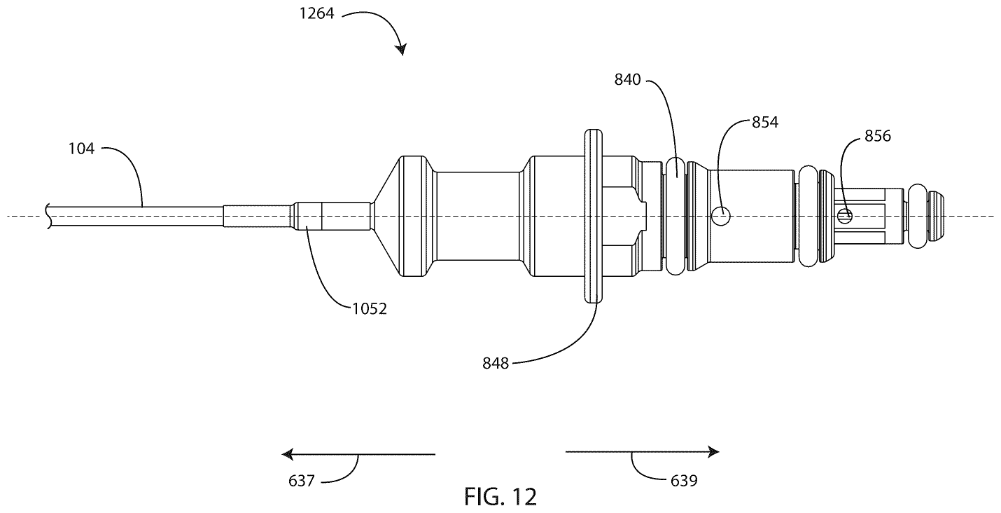

In various embodiments, the second connector piece 1060 can define a protrusion 1062. The protrusion 1062 of the second connector piece 1060 is configured to extend within a cavity 1064 defined within the first connector piece 1058. The second connector piece 1060 can also include a second cavity 1066. In various embodiments, the second cavity 1066 can be in fluid communication with conduit 868. For instance, fluid conduit 868 may form a portion of the second cavity 1066. Fluid conduit 868 is configured to receive the working gas from the handle 102 and deliver it to the shaft 104 by enclosing the supply tube 324. The fluid conduit 868 may include an interior space 1067 surrounded by an inner surface 1069. In various embodiments, the inner surface 1069 is configured to seal to an outer surface of the supply tube near the proximal end of the supply tube. The seal area between the supply tube 324 and the inner surface 1069 of the fluid conduit 868 is shown in FIG. 12.

The connector can further define a first tube fitting 1052 at a distal end. The connector can further define a second tube fitting 1063. The second tube fitting 1063 may form part of or extend from the protrusion 1062 of the second connector piece 1060 and be disposed within cavity 1064 of the first connector piece 1058. In various embodiments, the first tube fitting 1052 and the second tube fitting 1063 are each configured to attach to portions of the shaft 104. The protrusion 1062 includes an inner surface 1070, and the inner surface 1070 is configured to seal to an outer surface of the return tube near the proximal end of the return tube. The seal area between the return tube 326 and the inner surface 1070 of the protrusion 1062 is shown in FIG. 12. In various embodiments, the shaft-handle connector 103 is constructed from any suitable material or materials such as flexible metals, polymers, composites, or the like. In an embodiment, the shaft-handle connector 103 is constructed from Nitinol (NiTi), stainless steel, or the like.

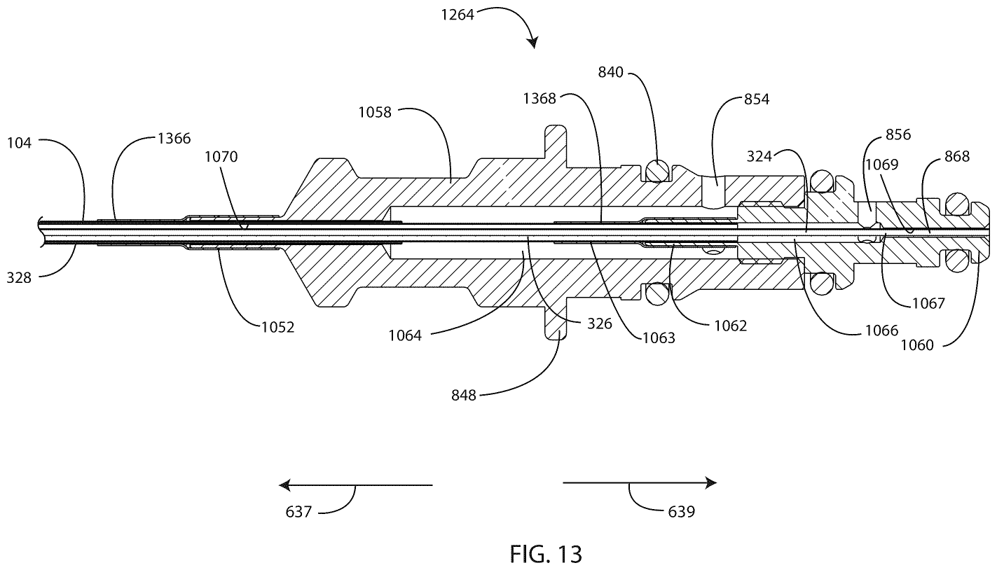

### Catheter Assembly (FIGS. 12-13)

Referring now to FIG. 12-13, various views of a catheter assembly are shown herein. FIG. 12 is a schematic view of the catheter assembly in accordance with various embodiments herein. FIG. 13 is a cross sectional view of the catheter assembly in accordance with various embodiments herein. In various embodiments, the catheter assembly 1264 can include the shaft 104 and the shaft-handle connector 103 of a cryoablation system 100. In some embodiments, the catheter assembly 1264 includes the components of the cryoablation system that are to be replaced each time a cryoablation procedure is performed.

In various embodiments, the shaft 104 can include a supply tube 324 extending along a portion of a length of the shaft. The supply tube can be surrounded by a return tube 326 along a portion of the length of the supply tube. The return tube 326 can be surrounded by an insulating shaft 328 along the insulated zone 105 of the shaft. In the example of FIGS. 12-13 the supply tube 324 terminates furthest in the proximal direction 639 of the cryoablation system 100 and the insulating shaft 328 terminates furthest in the distal direction 637 of the cryoablation system. In alternate configurations, the various layers of shaft 104 may terminate along the same location of the cryoablation system 100.

In various embodiments, the shaft-handle connector 103 is configured to form a seal around an outer surface the insulating shaft 328. In the example of FIGS. 12-13, the first tube fitting 1052 of the shaft-handle connector 103 is configured to seal around the proximal end of the insulating shaft 328. In some embodiments the insulating shaft 328 may be permanently connected to the shaft-handle connector 103. For instance, a proximal portion of the insulating shaft 328 may fit inside of the shaft-handle connector 103 and a portion 1366 of the first tube fitting 1052 can be melted and reflowed (or joined by another suitable means) over the outer surface of the insulating shaft 328 to form a seal between the insulating shaft 328 and the shaft-handle connector 103. In alterative embodiments, the insulating shaft 328 may be removably connected to the shaft-handle connector 103 using any suitable fastening means.

In various embodiments, the shaft-handle connector 103 is configured to form a seal around an outer surface the return tube 326. As shown in the example of FIG. 13, an inner surface 1070 of the second tube fitting 1063 and/or the protrusion 1062 of the shaft-handle connector 103 is configured to seal around the proximal end of the return tube 326. In some embodiments the return tube 326 may be permanently connected to the shaft-handle connector 103. For instance, a proximal portion of the return tube 326 may fit inside of the shaft-handle connector 103 and a portion 1368 of the second tube fitting 1063 can be melted and reflowed (or joined by another suitable means) over the outer surface of return tube 326 to form a seal between the return tube 326 and the shaft-handle connector 103. In alterative embodiments, the return tube 326 may be removably connected to the shaft-handle connector 103 using any suitable fastening means.

In various embodiments, the shaft-handle connector 103 is configured to form a seal around an outer surface of the supply tube 324. In the example of FIGS. 12-13, fluid conduit 868 (which may form a portion of the second cavity 1066) of the shaft-handle connector 103 is configured to seal around the proximal end of the supply tube 324. The fluid conduit 868 may include an interior space 1067 surrounded by an inner surface 1069 and the inner surface 1069 is configured to seal to an outer surface of the supply tube 324. In some embodiments the supply tube 324 may be permanently connected to the shaft-handle connector 103. For instance, a proximal portion of the supply tube 324 may fit inside of the shaft handle connector and can be permanently joined (e.g., by soldering, brazing, or the like) to the fluid conduit 868 of the shaft-handle connector 103. In alterative embodiments, the supply tube 324 may be removably connected to the shaft-handle connector 103 using any suitable fastening means.

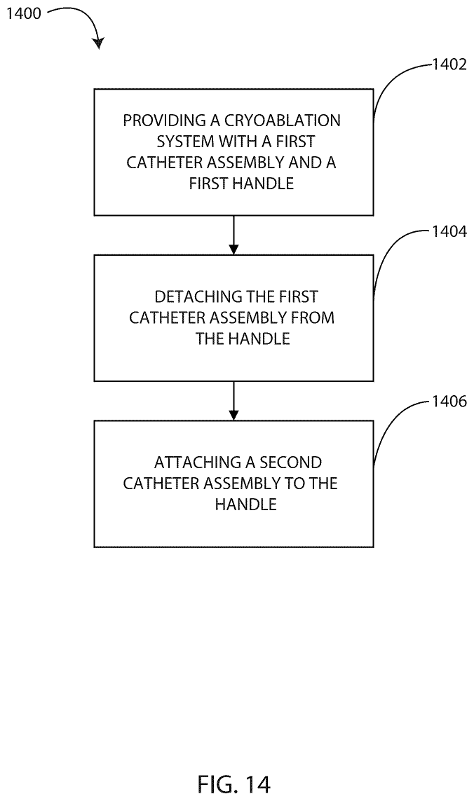

### Method of Operating a Cryoablation System (FIG. 14)

Many different methods are contemplated herein, including, but not limited to, methods of making, methods of using, and the like. Aspects of system/device operation described elsewhere herein can be performed as operations of one or more methods in accordance with various embodiments herein.

Referring now to FIG. 14, a method 1400 of operating a cryoablation system is described herein. The method 1400 can include the step 1402 of providing a cryoablation system. In various embodiments, the cryoablation system can include a pre-cooler gas circuit, a working gas circuit that is isolated from the pre-cooler gas circuit, and a vacuum chamber that is isolated from the working gas circuit and the pre-cooler gas circuit. The cryoablation system can further include a first catheter assembly. The first catheter assembly can include a first shaft and a first shaft-handle connector. The first shaft can include a first working gas expansion chamber, The cryoablation system can further include handle having a handle portion of the pre-cooler gas circuit and a handle portion of the working gas circuit that is isolated from the handle portion of the pre-cooler gas circuit. In various embodiments, the proximal end of the first shaft is configured to connect to the first shaft-handle connector.

The method 1400 can include the step 1404 of detaching the first catheter assembly from the handle. In various embodiments, the first shaft-handle connector removably attaches the proximal end of the first shaft to a distal end of the handle. In an embodiment, the first shaft can be removably attached to the handle by the first shaft-handle connector such that the first shaft can be removed from the handle without damaging the handle. Moreover, the first catheter assembly can be removed from the handle without inhibiting the ability of the handle to isolate the pre-cooler gas circuit, working gas circuit, vacuum chamber from one and other when a catheter assembly is attached to the handle.

In the example of FIGS. 6-8, the shaft-handle connector 103 may include a fastener 638 and the shaft 104 can be removed from the handle 102 by releasing the fastener (e.g., by unscrewing the fastener from the handle) and detaching the shaft from the handle. In various embodiments the step 1404 of detaching the catheter assembly 1264 from the handle 102 can be performed after each time the cryoablation system is used, for instance when a cryoablation procedure is performed on a patient.

The method 1400 can include the step 1406 of attaching a second catheter assembly to the handle. In various embodiments, the second catheter assembly comprises a second shaft and a second shaft-handle connector. The second shaft can include a second working gas expansion chamber. In various embodiments, step 1406 can include attaching the second shaft to the second shaft-handle to form a second catheter assembly and removably attaching the second catheter assembly to the handle. While the second catheter assembly can be assembled and attached to the handle using any suitable sequence of steps, one exemplary sequence is described in detail below.

In the example of FIGS. 12-13, the shaft-handle connector 103 can include a first connector piece 1058 and a second connector piece 1060. A protrusion 1062 of the second connector piece 1060 is configured to extend within a cavity 1064 defined within the first connector piece 1058. In such an embodiment, to attach the shaft 104 to the shaft-handle connector 103, the insulating shaft 328 can first be joined to the first connector piece 1058. For instance, a proximal portion of the insulating shaft 328 may fit inside of the shaft-handle connector 103 and a portion 1366 of the first tube fitting 1052 can be melted (or joined by another suitable means) over the outer surface of the insulating shaft 328 to form a seal between the insulating shaft 328 and the shaft-handle connector 103.

After attaching the insulating shaft 328 to the first connector piece 1058 the return tube 326 and the supply tube 324 can be joined to the second connector piece 1060 and the first connector piece 1058 can be attached to the second connector piece 1060. In an embodiment, the proximal portion of the return tube 326 may fit inside of the shaft-handle connector 103 and a portion 1368 of the second tube fitting 1063 can be melted (or joined by another suitable means) over the outer surface of return tube 326 to form a seal between the return tube 326 and the shaft-handle connector 103. In an embodiment, a fluid conduit 868 of the shaft-handle connector 103 is configured to seal around the proximal end of the supply tube 324. The first connector piece 1058 can then be securely attached to the second connector piece 1060 by any suitable means, such as an interference fit, or the like.

After assembling the second catheter assembly, the second catheter assembly can be removably attached to the distal end of the handle and secured to the handle using a fastener, or the like.

The concepts described herein can be applied in the context of and used in connection with cryoablation systems and components described in the following four U.S. nonprovisional patent applications, which are filed on the even date herewith, which are incorporated by reference herein in their entireties: U.S. Nonprovisional patent application Ser. No. ______, titled “Cryoablation Catheter Shaft Construction,” having attorney docket number 115.0421USU1; U.S. Nonprovisional patent application Ser. No. ______, titled “Safety Devices for Cryoablation Probe,” having attorney docket number 115.0422USU1; U.S. Nonprovisional patent application Ser. No. ______, titled “Delivery Systems for Cryoablation Device,” having attorney docket number 115.0424USU1; and U.S. Nonprovisional patent application Ser. No. ______, titled “Delivery Systems for Cryoablation Device,” having attorney docket number 115.0438US01.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cryoablation system comprising:
a working gas circuit;
a vacuum chamber isolated from the working gas circuit;
a shaft, the shaft comprising:
an insulated zone along a proximal length of the shaft, comprising a vacuum chamber shaft portion and an insulated portion of the working gas circuit, wherein the vacuum chamber shaft portion surrounds and is isolated from the insulated portion of the working gas circuit; and
a working gas expansion chamber distal to the insulated zone, wherein the working gas expansion chamber comprises an expansion portion of the working gas circuit;
a handle comprising, a handle portion of the vacuum chamber, and a handle portion of the working gas circuit; and
a shaft-handle connector, wherein a proximal end of the shaft connects to the shaft-handle connector, wherein the shaft-handle connector is configured to removably attach the proximal end of the shaft to a distal end of the handle.

2. The cryoablation system of claim 1, wherein the shaft comprises a supply tube extending along a portion of a length of the shaft, wherein the supply tube is surrounded by an return tube along a portion of a length of the supply tube, wherein the return tube is surrounded by an insulating shaft along the insulated zone of the shaft, wherein the shaft-handle connector is configured to form a seal around an outer surface the insulating shaft.

3. The cryoablation system of claim 2, wherein the shaft-handle connector comprises a first connector piece and a second connector piece, wherein a protrusion of the second connector piece is configured to extend within a cavity defined within the first connector piece.

4. The cryoablation system of claim 3, wherein an inner surface of the protrusion of the second connector piece of the shaft-handle connector is configured to form a seal around an outer surface of the return tube.

5. The cryoablation system of claim 3, wherein the second connector piece of the shaft-handle connector comprises an interior space and an inner surface of the interior space is configured to form a seal around an outer surface of the supply tube.

6. The cryoablation system of claim 1, wherein an inner surface of the handle is configured to form a seal around an outer surface of the shaft-handle connector.

7. The cryoablation system of claim 1, wherein the shaft can be removed from the handle without causing any damage to an ability of the handle isolate the handle portion of the working gas circuit and isolate the handle portion of the vacuum chamber.

8. The cryoablation system of claim 1, wherein the shaft-handle connector comprises a connector portion of the vacuum chamber, wherein the shaft-handle connector defines one or more openings in fluid communication with the connector portion of the vacuum chamber and is configured to connect to a vacuum chamber portion of the handle.

9. The cryoablation system of claim 1, wherein the shaft-handle connector defines one or more openings through which a return portion of the working gas circuit runs between the handle and the shaft-handle connector.

10. The cryoablation system of claim 1, wherein the cryoablation system further comprises a pre-cooler gas circuit isolated from the working gas circuit and the vacuum circuit, wherein the handle comprises a handle portion of the pre-cooler gas circuit, wherein the pre-cooler gas circuit is configured to supply a pre-cooler gas from a high-pressure cryogenic gas source to the handle, the pre-cooler gas circuit comprising a pre-cooler Joule-Thomson orifice where the pre-cooler gas enters a pre-cooler expansion chamber.

11. The cryoablation system of claim 1, wherein the working gas circuit is configured to supply a working gas from a high-pressure cryogenic gas source to the working gas expansion chamber, the working gas circuit comprising a working gas Joule-Thomson orifice where the working gas enters the working gas expansion chamber.

12. A cryoablation system comprising:
a working gas circuit;
a vacuum chamber isolated from the working gas circuit;
a shaft, the shaft comprising along a length of the shaft:
an insulated zone along a proximal length of the shaft, comprising a vacuum chamber shaft portion and an insulated portion of the working gas circuit, wherein the vacuum chamber shaft portion surrounds and is isolated from the insulated portion of the working gas circuit; and
a working gas expansion chamber distal to the insulated zone, wherein the working gas expansion chamber comprises an expansion portion of the working gas circuit; and
a shaft-handle connector, wherein a proximal end of the shaft connects to the shaft-handle connector, wherein the shaft-handle connector is configured to removably attach the proximal end of the shaft to a distal end of a handle, wherein the shaft-handle connector further comprises:
a working gas connector structure configured to make a sealed connection to a working gas supply passage in the handle and a working gas exhaust passage in the handle;
a vacuum connector structure configured to make a sealed connection to a vacuum chamber portion of the handle; and
wherein the shaft-handle connector comprises a connector portion of the vacuum chamber isolated from a connector portion of the working gas circuit.

13. The cryoablation system of claim 12, wherein the shaft comprises a supply tube extending along a portion of a length of the shaft, wherein the supply tube is surrounded by an return tube along a portion of a length of the supply tube, wherein the return tube is surrounded by an insulating shaft along the insulated zone of the shaft, wherein the shaft-handle connector is configured to form a seal around an outer surface of the insulating shaft.

14. The cryoablation system of claim 13, wherein the shaft-handle connector comprises a first piece and a second piece, wherein a protrusion of the second piece is configured to extend within a cavity defined within the first piece.

15. The cryoablation system of claim 14, wherein an inner surface of the protrusion of the second piece of the shaft-handle connector is configured to form a seal around an outer surface of the return tube.

16. The cryoablation system of claim 14, wherein the second piece of the shaft-handle connector comprises an interior space and an inner surface of the interior space is configured to form a seal around an outer surface of the supply tube.

17. The cryoablation system of claim 12, wherein the shaft-handle connector comprises a connector portion of the vacuum chamber, wherein the shaft-handle connector defines one or more openings in fluid communication with the connector portion of the vacuum chamber and is configured to connect to a vacuum chamber portion of the handle.

18. The cryoablation system of claim 12, wherein the shaft-handle connector defines one or more openings through which a return portion of the working gas circuit runs between the handle and the shaft-handle connector.

19. The cryoablation system of claim 12, wherein the working gas circuit is configured to supply a working gas from a high-pressure cryogenic gas source to the working gas expansion chamber, the working gas circuit comprising a working gas Joule-Thomson orifice where the working gas enters the working gas expansion chamber.

20. A method of operating a cryoablation system comprising:
providing a cryoablation system, the cryoablation system comprising:
a working gas circuit;
a first catheter assembly comprising a first shaft and a first shaft-handle connector, the first shaft comprising a first working gas expansion chamber;
a handle comprising a handle portion of the working gas circuit; and
wherein a proximal end of the first shaft connects to the first shaft-handle connector, wherein the first shaft-handle connector removably attaches the proximal end of the first shaft to a distal end of the handle;
detaching the first catheter assembly from the handle;
attaching a second catheter assembly to the handle, wherein the second catheter assembly comprises a second shaft and a second shaft-handle connector, the second shaft comprising a second working gas expansion chamber, wherein a proximal end of the second shaft connects to the second shaft-handle connector, wherein the second shaft-handle connector is configured to removably attach the proximal end of the second shaft to a distal end of the handle.
